(19) United States
(12) Patent Application Publication
(10) Pub. No.: US 2025/0327115 A1
(43) Pub. Date: Oct. 23, 2025

(54) REACTION BUFFER COMPOSITIONS AND METHODS FOR DNA AMPLIFICATION AND SEQUENCING

(71) Applicant: NUCLEIX LTD., Rehovot (IL)

(72) Inventor: Revital KNIRSH, Rosh HaAyin (IL)

(73) Assignee: NUCLEIX LTD., Rehovot (IL)

(21) Appl. No.: 18/868,117

(22) PCT Filed: May 22, 2023
(86) PCT No.: PCT/IL2023/050519

§ 371 Date: Nov. 21, 2024

(30) Foreign Application Priority Data
May 22, 2022 (IL) 293201

(51) Int. Cl. C12Q 1/683; C12Q 1/6858; C12Q 1/686; C12Q 1/6886

(52) U.S. Cl. CPC C12Q 1/683; C12Q 1/6858; C12Q 1/686; C12Q 1/6886; C12Q 2600/154

(57) ABSTRACT

Compositions and methods for DNA amplification following a DNA digestion reaction are provided. In particular embodiments, reaction buffers comprising a chelating agent are provided. The provided reaction buffers obviate the need for a dilution and/or a purification step between the DNA digestion and the DNA amplification.

### FIELD OF THE INVENTION

The present invention relates to compositions and methods for DNA amplification following a DNA digestion reaction. In particular, the present invention provides reaction buffers comprising a chelating agent which obviate the requirement of dilution and/or a purification step between the DNA digestion and the DNA amplification.

### BACKGROUND OF THE INVENTION

DNA screening and analysis requires several consecutive steps of activity of various enzymes and compounds. Different enzymes often need completely different conditions and/or presence of additional components for optimal activity. In some cases, a ‘universal’ buffer may be used in which, although not optimal, the different enzymes can be used with sufficient activity. In other cases, additional components are added between the different steps in order to adapt the conditions to the reactions. In some cases, the required conditions are too different and a full purification step is needed between reactions in order to adjust the conditions.

Genetic and epigenetic changes are known to occur in many types of cancer, including mutations, DNA methylation changes (e.g., hypomethylation of isolated CpGs and hypermethylation occurring mostly at CpG islands), copy number variation and more. For example, hypermethylation of CpG islands in the promotor regions of tumor suppressor genes, leading to gene silencing, has been studied extensively and demonstrated in many different types of cancer.

Common methods for identifying genetic and epigenetic changes involve the use of restriction enzymes whose activity varies according to these changes. The DNA is fragmented as a function of the genetic or epigenetic phenotype. PCR amplification is usually the next step for simple and accurate analyzing of the fragmented DNA. In some cases, the methods include a sequencing step. The different steps require certain conditions and reaction mixtures for optimal results.

Tumors release DNA fragments, or “cell-free DNA”, into body fluids and consequently genetic and epigenetic changes of tumor derived DNA molecules can be detected in “liquid biopsies” obtained from body fluids such as blood plasma and urine. In contrast to traditional biopsies, liquid biopsies are non-invasive and may better represent the full genetic spectrum of tumor sub-clones. Consequently, detection of genetic and epigenetic changes associated with cancer in liquid biopsies holds great promise for early detection, prognosis, and therapeutic surveillance. However, in order to detect tumor derived DNA in liquid biopsies, ultra-sensitive biochemical methods are required, as the concentration of cell-free DNA in biological fluids may be low, and furthermore because the tumor DNA can be present in extremely low quantities in relation to the large background of normal DNA.

Several techniques have been developed for detection of methylated DNA molecules in liquid biopsies-based digestion reactions by restriction enzymes, followed by quantitative PCR or DNA sequencing to detect methylation changes.

Brunner et al. (2009, Genome Res., 19(6): 1044-1056), report Methyl-seq, a method that assays DNA methylation at more than 90,000 regions throughout the genome. Methyl-seq combines DNA digestion by a methyl-sensitive enzyme with next-generation (next-gen) DNA sequencing technology.

Jelinek et al. (2012. Epigenetics, 7:12, 1368-1378), report a method entitled Digital Restriction Enzyme Analysis of Methylation (DREAM), which is based on next generation sequencing analysis of methylation-specific signatures created by sequential digestion of genomic DNA with a pair of neoschizomeric restriction enzymes that recognize the same sequence: Sma, a methylation-sensitive enzyme, and XmaI, a methylation-insensitive enzyme.

Marsh et al. (2016, Front Genet., 7:191), report a quantification methodology for computationally reconstructing site-specific CpG methylation status from next generation sequencing (NGS) data using methyl-sensitive restriction endonucleases (MSRE).

Tanaka et al. (2020, Analytical Biochemistry, 609: 113977), report an approach combining methylation-sensitive restriction enzyme (MSRE) and next-generation sequencing (NGS) to identify differentially methylated regions between chorionic villi (CV) and maternal blood cells (MBC).

WO 2011/070441, WO 2017/006317, WO 2019/142193 and WO 2020/188561, assigned to the Applicant of the present invention, disclose methods for detecting methylation changes in DNA samples.

It would be highly beneficial to have methods and reaction buffers that can improve the efficiency of DNA processing and analysis, that prevent DNA loss and sequencing noise.

### SUMMARY OF THE INVENTION

The present invention provides reaction mixtures and methods for DNA processing, advantageous for assays comprising DNA digestion followed by DNA amplification and DNA sequencing. The methods of the present invention comprise the addition of a chelating agent, such as EDTA, to the reaction mixture.

A large number of restriction-modification (R-M) systems have been discovered and well characterized during the past few decades. Based on the cutting position, recognition sequence, cleavage requirements, and subunit structure, R-M systems are mainly classified into four types I, II, III, and IV. The type II R-M systems are the most abundant group of enzymes; they produce double-stranded DNA cleavage within or close the recognition sequence which consists of 4- to 8-defined nucleotides that can be symmetric, asymmetric, or degenerate. Most of type II restriction endonucleases show an absolute requirement for divalent metal ions to catalyze in a charge repulsive, polyanionic context the cleavage of the phosphodiester bond, which is one of the most stable bonds in biochemistry. Although the physiological metal ion for the bacterial enzymes appears to be the magnesium, they can utilize a variety of divalent cations for in vitro DNA cleavage reaction, including Mn²⁺, Ca²⁺, Fe²⁺, Co²⁺, Ni²⁺, Zn²⁺, or Cd²⁺, depending on the enzyme. X-ray crystallographic analysis of type II restrictions enzymes in different metal-bound states has revealed two DNA cleavage mechanisms in which one or two metal ions are involved.

It is now disclosed that the addition of a chelating agent following a restriction enzyme digestion step enables the use of high concentrations of divalent cations in the digestion reaction, an amount that is suitable for the restriction enzymes. Advantageously, the addition of the chelating agent makes the removal or dilution of excess divalent cations for the amplification step, unnecessary. Moreover and unexpectedly, it is now disclosed that the addition of a chelating agent at the concentrations disclosed herein does not impair the amplification step or the subsequent sequencing.

The methods and systems of the present invention in some embodiments involve digestion of a DNA sample with methylation-sensitive or methylation-dependent restriction enzymes, followed by DNA amplification. In some embodiments, the DNA digestion is followed by DNA amplification and sequencing.

The present invention further discloses improved methods for determining methylation values for genomic loci of interest. Methylation analysis according to the present invention is carried out for restriction loci, namely, restriction sites of the restriction enzyme(s) used in the assay.

According to one aspect, the present invention provides a method for profiling methylation of a DNA sample from a subject, the method comprising:
(i) subjecting the DNA sample to digestion with at least one methylation-sensitive restriction endonuclease in the presence of an amount of divalent cations sufficient to support cleavage of the DNA sample by the at least one methylation-sensitive restriction endonuclease, to obtain restriction endonuclease-treated DNA in which methylated sites are intact and unmethylated sites are cut;
(ii) adding a chelating agent to reduce the availability of the divalent cations by chelating the divalent cations; and
(iii) amplifying from the restriction endonuclease-treated DNA at least one restriction locus, wherein the amplification is carried out in a reaction mix comprising a chelating agent and a divalent cation at a molar ratio of between 1:20 to 2:1, thereby generating an amplification product for said locus.

According to some embodiments, the DNA sample digestion with at least one methylation-sensitive restriction endonuclease is carried out in a reaction mix comprising 8-20 mM divalent cations, most preferably magnesium. According to some embodiments, the restriction enzyme requires at least 8 mM, 9 mM, 10 mM, 11 mM, 12 mM, 13 mM, 14 mM, 15 mM, 16 mM, 17 mM, 18 mM, 19 mM, 20 mM or more divalent cations for its activity. According to certain embodiments, the DNA sample digestion with at least one methylation-sensitive restriction endonuclease is carried out in a reaction mix comprising at least 8 mM, 9 mM, 10 mM, 11 mM, 12 mM, 13 mM, 14 mM, or 15 mM divalent cations, most preferably magnesium. According to certain exemplary embodiment, the divalent cations are magnesium (Mg²⁺). According to certain exemplary embodiments, the DNA sample digestion with at least one methylation-sensitive restriction endonuclease is carried out in a reaction mix comprising about 10 mM magnesium.

According to some embodiments, the divalent cation is selected from the group consisting of Mg²⁺, Mn²⁺, Ca²⁺, Fe²⁺, Co²⁺, Ni²⁺, Zn²⁺, or Cd²⁺. According to certain embodiments, the divalent cation is magnesium.

According to some embodiments, the restriction enzyme requires at least 8, 9, 10, 11, 12, 13, 14, 15, 16, 17, 18, 19, 20 mM or more magnesium for its activity. Each possibility represents a separate embodiment of the invention.

In certain embodiments, the divalent cations are magnesium ions, and the chelating agent is selected for its ability to chelate magnesium. By way of example, the chelating agent comprises one or both of EDTA and EGTA. According to some embodiments, the chelating agent is EDTA. According to other embodiments, the chelating agent is EGTA.

According to some embodiments, the amplification is carried out in a reaction mix comprising a chelating agent and a divalent cation at a molar ratio of between 1:10 to 2:1. According to some embodiments, the amplification is carried out in a reaction mix comprising a chelating agent and a divalent cation at a molar ratio of between 1:5 to 2:1, 1:2 to 2:1, or 1:2 to 1:1. According to certain embodiments, the amplification is carried out in a reaction mix comprising a chelating agent and a divalent cation at a molar ratio of between 1:2 to 2:1. According to certain embodiments, the amplification is carried out in a reaction mix comprising about 3 mM chelating agent and 4 mM divalent cations. According to additional embodiments, the amplification is carried out in a reaction mix comprising about 4 mM chelating agent and 4 mM divalent cations.

According to some embodiments, the amplification step is carried out in a reaction mix comprising between 0.5-5 mM of the chelating agent. According to some embodiments, the amplification step is carried out in a reaction mix comprising between 1-4, 2-4, 2-5, or 3-4 mM of the chelating agent.

According to some embodiments, adding a chelating agent is performed by adding the restriction endonuclease-treated DNA of step (i) to reaction mix comprising the chelating agent.

According to some embodiments, the digestion step is carried out in the absence of a chelating agent. According to other embodiments, the digestion step is carried out in a reaction mix having less than 2 mM, 1.5 mM, 1 mM or 0.5 mM chelating agent.

According to some embodiments, the DNA sample is a cell-free DNA sample.

According to some embodiments, the DNA is cell-free DNA extracted from a biological fluid sample. In some embodiments, the biological fluid sample is plasma, serum or urine. Each possibility of the biological sample is a separate embodiment of the present invention. According to some embodiments, the sample is a plasma sample.

According to some embodiments, an amount of cell-free DNA comprising 6,000 haploid equivalents is sufficient for the methods disclosed herein. According to some embodiments, the cell-free DNA is plasma cell-free DNA, and the amount of the cell-free DNA is an amount obtained from 8-10 ml of blood. According to some embodiments, the amount of cell-free DNA is between 10-400 ng. According to some embodiments, the amount of cell-free DNA is between 10-250 ng. According to some embodiments, the amount of cell-free DNA is between 10-200 ng. According to additional embodiments, the amount of cell-free DNA is between 20-100 ng.

According to some embodiments, the DNA is DNA extracted from a tumor sample.

According to some embodiments, the at least one methylation-sensitive restriction endonuclease is a plurality of methylation-sensitive restriction endonucleases.

According to some embodiments, the methylation-sensitive restriction endonuclease is selected from the group consisting of: AatII, Acc65I, AccI, AciI, AfeI, AgeI, ApaI, ApaLI, AscI, AsiSI, AvaI, AvaII, BaeI, BanI, BbeI, BceAI, BcgI, BfuCI, BglI, BmgBI, BsaAI, BsaBI, BsaHI, BsaI, BseYI, BsiEI, BsiWI, BslI, BsmAI, BsmBI, BsmFI, BspDI, BsrBI, BsrFI, BssHII, BssKI, BstAPI, BstBI, BstUI, BstZ17I, Cac8I, ClaI, DpnI, DrdI, EaeI, EagI, EagI-HF, EciI, EcoRI, EcoRI-HF, FauI, Fnu4HI, FseI, FspI, HaeII, HgaI, HhaI, HincII, HinfI, HinP1I, HpaI, HpaII, Hpy166ii, Hpy188iii, Hpy99I, HpyCH4IV, KasI, MluI, MmeI, MspA1I, MwoI, NaeI, NgoMIV, NheI-HF, NheI, NlaIV, NotI, NotI-HF, NruI, PaeR7I, PleI, PmeI, PmlI, PshAI, PspOMI, PvuI, RsaI, RsrII, SacII, SalI, SalI-HF, Sau3AI, Sau96I, ScrFI, SfiI, SfoI, SgrAI, SmaI, SnaBI, TfiI, TseI, TspMI, and ZraI. Each possibility represents a separate embodiment of the present invention. According to some embodiments, the at least one methylation-sensitive restriction endonuclease is selected from the group consisting of AciI, HinP1I and HhaI. According to some embodiments, the at least one methylation-sensitive restriction endonuclease comprises HinP1I. According to certain embodiments, the at least one methylation-sensitive restriction endonuclease comprises HhaI. In yet additional particular embodiments, the at least one methylation-sensitive restriction endonuclease comprises AciI. According to some embodiments, step (i) comprising digestion with the restriction enzymes HinP1I and HhaI.

According to some embodiments, the reaction mix is a reaction buffer comprising Tris-HCl. According to some embodiments, the reaction mix comprises dNTPs. According to some embodiments, the reaction mix comprises primers and/or probes.

According to some embodiments, the method comprises simultaneous amplification of more than one target sequence in the same reaction mixture.

According to some embodiments, the amplification step comprises a step of co-amplification of at least one restriction locus and a control locus, thereby generating an amplification product for each locus. According to certain embodiments, the control locus is a locus devoid of a recognition sequence of the methylation-sensitive restriction endonuclease.

According to some embodiments, step (iii) is performed using real-time PCR. According to certain embodiments, when step (iii) is performed using real-time PCR, the method further comprises adding fluorescent probes for assisting in detecting the amplification products.

According to some embodiments, the method comprises determining a signal intensity for each generated amplification product. According to certain embodiments, the method comprises a step of comparing a ratio between the signal intensities of the amplification products of each of said at least one restriction locus and the control locus to at least one reference ratio. According to some embodiments, the control locus is a locus devoid of a recognition sequence of the methylation-sensitive restriction endonuclease.

According to some embodiments, the ratio between the signal intensities of the amplification products of each of said at least one restriction locus and the control locus is calculated by determining the quantification cycle (Cq) for each locus and calculating the ratio as 2^{(Cq control locus-Cq restriction locus)}.

According to some embodiments, the DNA sample contains less than 10%, 8%, 6%, 4% or 2% single strand DNA (ssDNA) by weight. According to some embodiments, the DNA sample contains less than 1% ssDNA by weight. According to some embodiments, the DNA sample contains less than 0.1% ssDNA. According to some embodiments, the DNA sample contains less than 0.01% ssDNA or is free of ssDNA.

According to some embodiments, the method comprises a step of preparing a sequencing library.

According to some embodiments, the method comprising sequencing the library by a high-throughput sequencing method to provide sequencing data. According to certain embodiments, the method comprising determining from the sequencing data a methylation value for at least one restriction locus and optionally at least one additional genetic or epigenetic characteristic of the DNA sample, e.g., DNA mutation and copy number variation.

According to some embodiments, the at least one restriction locus is located within a CG-island.

According to some embodiments, the at least one restriction locus is a plurality of restriction loci.

According to some embodiments, the method for profiling methylation further comprises identifying the presence or absence of a disease in the subject based on the methylation profile of the DNA sample, by comparing the methylation profile of the DNA sample to one or more reference methylation profile.

According to some embodiments, the DNA sample is from a subject suspected of having the disease and/or a subject at risk of developing the disease, and the method comprises detecting methylation changes comprising determining whether the DNA sample is a healthy or disease DNA sample. According to some embodiments, the disease is cancer.

According to some embodiments, the method further comprises preparing a report in paper or electronic form based on the methylation profile and communicating the report to the subject and/or to a healthcare provider of the subject.

According to another aspect, the present invention provides a PCR reaction mix comprising between 0.5 mM and 50 mM chelating agent and a Taq polymerase.

According to some embodiments, the PCR reaction mix comprises dNTPs.

According to some embodiments, the PCR reaction mix comprising at least one primer. According to some embodiments, the PCR reaction mix comprising a plurality of primers. According to certain embodiments, the PCR reaction mix comprises probes.

According to some embodiments, the chelating agent is EDTA. According to other embodiments, the chelating agent is EGTA.

According to some embodiments, the PCR reaction mix comprising between 2-50, 4-40, 5-30, 5-20, or 10-20 mM of the chelating agent.

According to certain embodiments, the PCR reaction mix further comprises restriction enzyme digested DNA.

According to another aspect, the present invention provides a reaction mix for high-throughput sequencing comprising between 0.5 mM and 50 mM chelating agent.

According to some embodiments, the reaction mix comprises adapters and/or DNA ligation enzyme.

According to another aspect, the present invention provides a kit comprising a DNA amplification reaction mix comprising between 0.5 mM and 50 mM chelating agent and Taq polymerase.

According to some embodiments, the kit comprises at least one methylation-sensitive restriction endonuclease as described hereinabove. According to some embodiments, the kit comprises a DNA digestion reaction mix comprising magnesium.

According to another aspect, the present invention provides a method for profiling methylation of a DNA sample from a subject, the method comprising:
(i) subjecting the DNA sample to digestion with at least one methylation-sensitive restriction endonuclease or at least one methylation-dependent restriction endonuclease in the presence of an amount of divalent cations sufficient to support cleavage of the DNA sample by the at least one methylation-sensitive or methylation-dependent restriction endonuclease, to obtain restriction endonuclease-treated DNA;
(ii) adding a chelating agent to reduce the availability of the divalent cations by chelating the divalent cations; and
(iii) preparing a sequencing library from the restriction endonuclease-treated DNA, wherein preparing the sequencing library comprises introducing adapter sequences into DNA fragments in the restriction endonuclease-treated DNA using PCR amplification, and wherein the PCR amplification is carried out in a buffer comprising a chelating agent and a divalent cation at a molar ratio of between 1:20 to 2:1.

According to another aspect, the present invention provides a method for profiling methylation of a DNA sample from a subject, the method comprising:
(i) subjecting the DNA sample to digestion with at least one methylation-sensitive restriction endonuclease or at least one methylation-dependent restriction endonuclease in the presence of an amount of divalent cations sufficient to support cleavage of the DNA sample by the at least one methylation-sensitive or methylation-dependent restriction endonuclease, to obtain restriction endonuclease-treated DNA;
(ii) adding a chelating agent to reduce the availability of the divalent cations by chelating the divalent cations;
(iii) amplifying from the restriction endonuclease-treated DNA at least one restriction locus, wherein the amplification is carried out in a reaction mix comprising a chelating agent and a divalent cation at a molar ratio of between 1:20 to 2:1, thereby generating an amplification product for said locus; and
(iv) preparing a sequencing library from the amplification products, wherein preparing the sequencing library comprises adding (e.g., ligating) sequencing adapters to the amplification products.

According to another aspect, the present invention provides a method for analyzing a DNA sample comprising:
(i) subjecting the DNA sample to digestion with at least one methylation-sensitive or methylation-dependent restriction endonuclease in the presence of an amount of divalent cations sufficient to support cleavage of the DNA sample by the at least one methylation-sensitive or methylation-dependent restriction endonuclease, to obtain restriction endonuclease-treated DNA;
(ii) adding a chelating agent to reduce the availability of the divalent cations by chelating the divalent cations; and
(iii) amplifying from the restriction endonuclease-treated DNA at least one restriction locus, wherein the amplification is carried out in a reaction mix comprising a chelating agent and divalent cations at a molar ratio of between 1:20 to 2:1, thereby generating an amplification product for said locus.

According to some embodiments, the amplification is carried out in a reaction mix comprising a chelating agent and divalent cations at a molar ratio of between 1:20 to 2:1.

According to some embodiments, the amplification step is carried out in a reaction mix comprising between 0.5-5 mM of the chelating agent. According to some embodiments, the amplification step is carried out in a reaction mix comprising between 1-4, 2-4, 2-5, or 3-4 mM of the chelating agent. According to certain embodiments, the amplification step is carried out in a reaction mix comprising between 1, 2, or 3 mM and up to 4.5, 4.6, 4.7, 4.8, or 4.9 mM of the chelating agent. Each possibility represents a separate embodiment of the invention.

According to some embodiments, the preparation of the sequencing library is carried out in a buffer comprising 0.5-5 mM of a chelating agent.

According to some embodiments, the method comprises a step of sequencing the library by a high-throughput sequencing method to obtain sequencing data.

According to some embodiments, an amount of cell-free DNA comprising 6,000 haploid equivalents is sufficient for the methods disclosed herein.

According to some embodiments, the cell-free DNA is plasma cell-free DNA, and the amount of the cell-free DNA is an amount obtained from 8-10 ml of blood.

According to some embodiments, the amount of cell-free DNA is between 5-500 ng. According to some embodiments, the amount of cell-free DNA is between 10-400 ng. According to some embodiments, the amount of cell-free DNA is between 10-250 ng. According to additional embodiments, the amount of cell-free DNA is between 20-100 ng.

According to some embodiments, the high-throughput sequencing is target-specific high-throughput sequencing.

According to certain embodiments, the method comprising determining from the sequencing data a methylation value for at least one restriction locus and optionally at least one additional genetic or epigenetic characteristic of the DNA sample, e.g., DNA mutation and copy number variation.

According to some embodiments, determining a methylation value for at least one restriction locus comprises: determining read counts and relative copy number between the at least one restriction and a control locus.

According to some embodiments, the at least one restriction locus is a plurality of restriction loci.

According to some embodiments, the at least one methylation-sensitive restriction endonuclease is a plurality of methylation-sensitive restriction endonucleases, and the digestion with the plurality of methylation-sensitive restriction endonucleases is a simultaneous digestion.

According to some embodiments, the step of subjecting the cell-free DNA sample to digestion with at least one methylation-sensitive restriction endonuclease further comprises determining digestion efficacy. In some embodiments, proceeding to preparing a sequencing library is carried out if the digestion efficacy is above a predefined threshold.

According to another aspect, the present invention provides a method for detecting cancer-related genetic and epigenetic changes in a cell-free DNA sample (cfDNA) from a subject, the method comprising: profiling methylation and optionally at least one additional genetic and epigenetic characteristics of the cfDNA sample as disclosed herein, to obtain a genetic and epigenetic profile of the cfDNA sample; and comparing the genetic and epigenetic profile of the cfDNA sample to one or more reference genetic and epigenetic profile selected from a cancer profile and a non-cancer profile, to detect cancer-associated genetic and epigenetic changes in the cfDNA sample.

According to some embodiments, the cell-free DNA sample is from a subject suspected of having cancer or at risk of having cancer, and the method further comprises administering to the subject active cancer surveillance and follow-up testing when cancer-associated changes are detected, the cancer surveillance and follow-up testing comprise one or more of blood tests, urine tests, cytology, imaging, endoscopy and biopsy.

According to a further aspect, the present invention provides a method for characterizing a cell-free DNA (cfDNA) sample of a subject suspected of having cancer or at risk of having cancer, the method comprising:
(a) subjecting the cell-free DNA sample to digestion with at least one methylation-sensitive restriction endonuclease in the presence of an amount of divalent cations sufficient to support cleavage of the DNA sample by the at least one methylation-sensitive restriction endonuclease, to obtain restriction endonuclease-treated DNA in which methylated sites are intact and unmethylated sites are cut;
(b) adding a chelating agent to reduce the availability of the divalent cations by chelating the divalent cations; and
   (i) amplifying from the restriction endonuclease-treated DNA at least one restriction locus, wherein the amplification is carried out in a reaction mix comprising a chelating agent and divalent cations at a molar ratio of between 1:20 to 2:1, thereby generating an amplification product for said locus; and optionally
   (ii) preparing a sequencing library from the amplification products.

According to another aspect, the present invention provides a method for genetic and epigenetic profiling of a DNA sample, the method comprising determining a methylation value for at least one restriction locus as disclosed herein, and further determining from the sequencing data at least one additional genetic or epigenetic characteristic of the DNA sample, e.g., DNA mutation and copy number variation.

According to a further aspect, the present invention provides a method for identifying genomic regions differentially methylated between a first and second source of DNA, the method comprising:
profiling methylation of at least one DNA sample from the first source as disclosed herein, to obtain a first DNA methylation profile;
profiling methylation of at least one DNA sample from the second source as disclosed herein, to obtain a second DNA methylation profile; and
comparing the first and second DNA methylation profiles to identify genomic regions differentially methylated between the first and second sources of DNA.

According to some embodiments, the first source of DNA is a cancer DNA and the second source of DNA is a non-cancer DNA. According to some embodiments, the first source of DNA is plasma cell-free DNA of a cancer patient and the second source of DNA is plasma cell-free DNA of one or more healthy individuals. In additional embodiments, the first and second sources of DNA are different stages of a cancer.

According to another aspect, the present invention provides a method for detecting methylation changes in a DNA sample, the method comprising: profiling methylation of the DNA sample as disclosed herein, to obtain a methylation profile of the DNA sample; and comparing the methylation profile of the DNA sample to one or more reference methylation profile to detect methylation changes in the DNA sample.

It is to be understood that any combination of each of the aspects and the embodiments disclosed herein is explicitly encompassed within the disclosure of the present invention.

Further embodiments and the full scope of applicability of the present invention will become apparent from and detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1. Agarose gel analysis of the products of PCR performed with or without EDTA. Arrows (i) indicate nonspecific PCR products. Arrows (ii) indicate specific PCR products.

### DETAILED DESCRIPTION OF THE INVENTION

The present invention relates to methods and compositions for profiling genetic and epigenetic characteristics of DNA samples, particularly cell-free DNA samples, using digestion of DNA with methylation-sensitive/methylation-dependent restriction enzymes followed by a step of adding a chelating agent; and DNA amplification, high throughput sequencing and analysis of sequence reads. Advantageously, the methods and compositions of the present invention enable working with very low amounts of DNA and receive vast amount of information, including methylation data, mutation data and more.

The methods of the invention comprise the addition of a chelating agent, preferably EDTA, that eliminates the need to remove or dilute the divalent cation, e.g., magnesium used in a DNA digestion step, before proceeding to an amplification step. This is in particular important when using low amounts of DNA and a purification step or dilution may impair results.

According to one aspect, the present invention provides a method for profiling methylation of a DNA sample from a subject, the method comprising:
(i) subjecting the DNA sample to digestion with at least one methylation-sensitive or methylation-dependent restriction endonuclease, to obtain restriction endonuclease-treated DNA;
(ii) adding a chelating agent; and
(iii) amplifying from the restriction endonuclease-treated DNA at least one restriction locus, wherein the amplification is carried out in a reaction mix comprising a chelating agent and divalent cations at a molar ratio of between 1:20 to 2:1.

According to an additional aspect, the present invention provides a method for analyzing a DNA sample from a subject, the method comprising:
(i) subjecting the DNA sample to digestion with at least one restriction endonuclease, to obtain restriction endonuclease-treated DNA;
(ii) adding a chelating agent; and
(iii) amplifying from the restriction endonuclease-treated DNA at least one restriction locus, wherein the amplification is carried out in a reaction mix comprising a chelating agent and divalent cations at a molar ratio of between 1:20 to 2:1, thereby generating an amplification product for said locus.

According to some embodiments, the restriction enzyme is a methylation-sensitive restriction enzyme. According to other embodiments, the restriction enzyme is a methylation-dependent restriction enzyme.

The terms “chelating agent” and “chelator” are used herein as known in the art and refer to a chemical compound that reacts with metal ions to form stable, water-soluble metal complexes. The added chelating agent is used herein to prevent deleterious effects of excess metals, in particular magnesium, on processes such as DNA amplification and sequencing.

According to some embodiments, the chelating agent is EDTA. According to other embodiments, the chelating agent is EGTA.

The term “reaction mix” as used herein refers to aqueous solutions or compositions that are suitable for carrying out the indicated reaction, such as PCR amplification, sequencing, DNA digestion, etc.

The term “buffer” as used herein, refers to aqueous solutions or compositions that resist changes in pH when acids or bases are added to the solution and are suitable for carrying out the indicated reaction, such as PCR amplification, DNA digestion, etc.

The term “amplification”, as used herein, refers to an increase in the number of copies of one or more particular nucleic acid target of interest. Amplification is typically performed by polymerase chain reaction (PCR) in the presence of a PCR reaction mixture which may include a suitable buffer supplemented with the DNA template, polymerase (usually Taq Polymerase), dNTPs, primers and probes (as appropriate), as known in the art.

An “amplification product” collectively refers to nucleic acid molecules of a particular target sequence that are generated and accumulated in an amplification reaction. The term generally refers to nucleic acid molecules generated by PCR using a given set of amplification primers.

As used herein, a “primer” defines an oligonucleotide which is capable of annealing to (hybridizing with) a target sequence, thereby creating a double stranded region which can serve as an initiation point for DNA synthesis under suitable conditions.

As used herein the term “plurality” refers to more than one, namely two or more.

### DNA Sample

A DNA sample for use according to the present invention may be obtained from any biological sample of a subject from which nucleic acids can be obtained, including biological fluid samples such as blood, plasma, serum, urine, cerebrospinal fluid, semen, stool, sputum and amniotic fluid. Each possibility represents a separate embodiment of the present invention. Biological samples also include tissue and organ samples.

A “subject” according to the present invention is typically a human subject. The subject may be suspected of having a certain disease. In some embodiments, the subject is diagnosed with a disease of interest. In other embodiments, the subject is a healthy subject that does not have the disease of interest. The subject may also be at risk of developing the disease, for example, based on previous history of the disease, genetic predisposition, and/or family history, and/or a subject who exhibits suspicious clinical signs of the disease and/or a subject that is suspected of having the disease based on other prior assay(s) e.g., based on testing of other biomarker(s). In some embodiments, the subject is at risk of recurrence of the disease. In some embodiments, the subject shows at least one symptom or characteristic of the disease. In other embodiments, the subject is asymptomatic.

According to some embodiments, the DNA sample is cell-free DNA extracted from a biological fluid sample. The term “cell-free DNA” (abbreviated “cfDNA”) refers to DNA molecules which are freely circulating in body fluids and are not contained within intact cells. The origin of cfDNA is not fully understood but believed to be related to apoptosis, necrosis and active release from cells. cfDNA is released by both normal and tumor cells. cfDNA is highly fragmented, with fragments typically ranging between 120-220 bps in length, mostly between 150-180 bps in length. It is to be understood that the term “cell-free DNA” as used herein refers to DNA which is already cell-free in the body of the subject. It is to be understood that for cell-free DNA samples, “restriction endonuclease-treated DNA” comprises fragments generated as a result of the digestion, and also natural cell-free DNA fragments, for example, cell-free DNA fragments that do not contain a recognition sequence of the enzyme(s) used in the assay and cell-free DNA fragments that contain one or more recognition sequences of the enzyme(s) that are all methylated and therefore not cut by the enzyme.

Alternatively, the DNA sample may be DNA extracted from cells, for example, DNA extracted from tissue or organ samples or from blood cells. Typically, cell lysis is required in order to extract the DNA. DNA may be obtained from tumor samples or from healthy tissues. A “tumor sample” as used herein encompasses a whole tumor resected by surgery or portions thereof. A “tumor sample” also encompasses a sample taken from a tumor by biopsy, and a sample taken from a lesion or a tissue suspected of being cancerous. Tumor samples for use according to the present invention include fresh tumor samples as well as frozen/preserved tumor samples.

According to some embodiments, the methods disclosed herein further comprise a step of sequencing. For DNA extracted from cells, a step of fragmenting the DNA into fragments suitable for high-throughput sequencing may be carried out before, after or during the digestion with the at least one methylation-sensitive or methylation-dependent restriction endonuclease according to the present invention, to simplify downstream processing and preparation of a sequencing library. Such fragmentation can be carried out, for example, using sonication, or using a restriction endonuclease which is insensitive to methylation, namely, cleaves its recognition sequence regardless of methylation status. It can also be carried out using a restriction endonuclease with a recognition sequence that does not include CG dinucleotides.

According to some embodiments, the fragmented DNA is analyzed directly by amplification of specific locus or loci.

Preferably, the DNA sample on which the methylation analysis is carried out is substantially free of single-stranded DNA (ssDNA). As used herein, “substantially free of ssDNA” or “substantially devoid of ssDNA” indicates a DNA sample in which less than 7% of the DNA is ssDNA, preferably less than 5% of the DNA is ssDNA, more preferably less than 1% of the DNA is ssDNA (namely, at least 99% of the DNA is double-stranded) (by number of molecules). In some embodiments, the DNA sample contains less than 0.1% ssDNA. In some embodiments, the DNA sample contains less than 0.01% ssDNA. In some embodiments, the DNA sample contains no ssDNA (free of ssDNA). Extraction of DNA to obtain a DNA sample substantially free of ssDNA is described, for example, in WO 2020/188561, assigned to the Applicant of the present invention. An exemplary kit for extracting cell-free DNA which is suitable for use with the method of the present invention is QIAamp® Circulating Nucleic Acid Kit (QIAGEN, Hilden, Germany). An exemplary kit for extracting DNA from cells is QIAamp® Blood Mini Kit.

### DNA Digestion

According to the present invention, following extraction (and optionally enrichment for regions of interest and/or fragmentation to reduce size) the DNA is subjected to digestion with at least one methylation-sensitive restriction endonuclease and/or at least one methylation-dependent restriction endonuclease, preferably with a plurality of methylation-sensitive restriction endonucleases (or a plurality of methylation-dependent restriction endonucleases) applied simultaneously. As used herein, “restriction endonucleases applied simultaneously” or “simultaneous digestion” means that the enzymes are present together in the reaction mixture in an active form, without inactivation of one prior to application of another.

For example, one, two, three, four or five methylation-sensitive and/or methylation-dependent restriction endonucleases may be used. Each number of endonucleases used in the assay represents a separate embodiment of the present invention.

According to some embodiments, the entire DNA that was extracted is used in the digestion step. In some embodiments, the DNA is not quantified prior to being subjected to digestion. In other embodiments, the DNA is quantified prior to digestion thereof. In some embodiments, the DNA is aliquoted into a first aliquot that is subjected to digestion and a second aliquot that is kept as an undigested control.

A “restriction endonuclease”, used herein interchangeably with a “restriction enzyme”, refers to an enzyme that cuts DNA at or near specific recognition sequences, also known as restriction sites. Restriction sites are usually 4 to 8 nucleotide long and are typically palindromic (i.e., reading in a certain direction, e.g. 5′ to 3′, on one strand is identical to the sequence in the same direction (5′ to 3′) on the complementary strand).

A “methylation-sensitive” restriction endonuclease is a restriction endonuclease that cleaves its recognition sequence only if it is unmethylated (while methylated sites remain intact). Thus, the extent of digestion of a DNA sample by a methylation-sensitive restriction endonuclease depends on the methylation level, where a higher methylation level protects from cleavage and accordingly results in less digestion. A DNA sample treated with a methylation-sensitive restriction endonuclease is characterized by intact methylated sites and cut unmethylated sites. It is to be understood that there is no need for 100% digestion efficiency and thus some unmethylated sites might remain intact. In some embodiments, the methods of the present invention comprise determining the digestion efficacy, and proceeding to preparing a sequencing library if the digestion efficacy is above a predefined threshold/level.

A “methylation-dependent” restriction endonuclease is a restriction endonuclease that cleaves its recognition sequence only if it is methylated (while unmethylated sites remain intact). Thus, the extent of digestion of a DNA sample by a methylation-dependent restriction endonuclease depends on the methylation level, where a higher methylation level results in more extensive digestion.

Methylation-sensitive restriction endonuclease(s) for use according to the present invention may be selected from the group consisting of: AatII, Acc65I, AccI, AciI, AfeI, AgeI, ApaI, ApaLI, AscI, AsiSI, AvaI, AvaII, BaeI, BanI, BbeI, BceAI, BcgI, BfuCI, BglI, BmgBI, BsaAI, BsaBI, BsaHI, BsaI, BseYI, BsiEI, BsiWI, BslI, BsmAI, BsmBI, BsmFI, BspDI, BsrBI, BsrFI, BssHII, BssKI, BstAPI, BstBI, BstUI, BstZ17I, Cac8I, ClaI, DpnI, DrdI, EaeI, EagI, EagI-HF, EciI, EcoRI, EcoRI-HF, FauI, Fnu4HI, FseI, FspI, HaeII, HgaI, HhaI, HincII, HinfI, HinP1I, HpaI, HpaII, Hpy166ii, Hpy188iii, Hpy99I, HpyCH4IV, KasI, MluI, MmeI, MspA1I, MwoI, NaeI, NgoMIV, NheI-HF, NheI, NlaIV, NotI, NotI-HF, NruI, PaeR7I, PleI, PmeI, PmlI, PshAI, PspOMI, PvuI, RsaI, RsrII, SacII, SalI, SalI-HF, Sau3AI, Sau96I, ScrFI, SfiI, SfoI, SgrAI, SmaI, SnaBI, TfiI, TseI, TspMI, and ZraI. Each possibility represents a separate embodiment of the present invention. In some particular embodiments, the at least one methylation-sensitive restriction endonuclease comprises HinP1I. In additional particular embodiments, the at least one methylation-sensitive restriction endonuclease comprises HhaI. In yet additional particular embodiments, the at least one methylation-sensitive restriction endonuclease comprises AciI.

Methylation-dependent restriction endonuclease(s) may be selected from the group consisting of: McrBC, McrA, and MrrA. Each possibility represents a separate embodiment of the present invention.

According to some embodiments, a DNA sample according to the present invention is subjected to digestion with a single methylation-sensitive restriction endonuclease. In some particular embodiments, the methylation-sensitive restriction endonuclease is HinP1I. In additional particular embodiments, the methylation-sensitive restriction endonuclease is HhaI. In additional embodiments, the DNA sample is subjected to digestion with two methylation-sensitive restriction endonucleases.

In some particular embodiments, the methylation-sensitive restriction endonucleases HinP1I and AciI are used.

In some embodiments, there is provided a method for profiling methylation of a DNA sample, the method comprising: subjecting the DNA sample to digestion with the methylation-sensitive restriction endonucleases HinP1I and AciI; and analyzing methylation of at least one restriction locus of HinP1I and/or at least one restriction locus of AciI, thereby profiling methylation of the DNA sample. In some embodiments, the method comprises subjecting the DNA sample to digestion with the methylation-sensitive restriction endonucleases HinP1I and AciI; and determining a level of methylated DNA and optionally a level of unmethylated DNA of at least one restriction locus of HinP1I and/or at least one restriction locus of AciI, thereby profiling methylation of the DNA sample. In some embodiments, the DNA sample is cell-free DNA extracted from a biological fluid.

In some embodiments, HinP1I and AciI at a ratio between 1:1 to 5:1 (enzyme units) (Hinp:AciI) are used with the methods and systems of the present invention, for example 2:1. 2.5:1, 3:1, 3.5:1, 4:1 and 4.5: 1 (enzyme units) (Hinp:AciI). Each possibility represents a separate embodiment of the present invention. In some embodiments, HinP1I and AciI at a ratio between 2:1 to 4.5:1 (enzyme units) (Hinp:AciI) are used with the methods and systems of the present invention.

The digestion is carried out in a reaction buffer that contains several components for optimal activity of the restriction enzymes. Reaction buffers may contain, for example, Tris-HCl, MgCl₂, NaCl, and 2-mercaptoethanol.

According to some embodiments, the restriction enzymes used herein requires at least 8, 9, 10, 11, 12 mM or more of MgCl₂ for their optimal activity. According to some embodiments, the DNA digestion reaction mix comprises 10 mM MgCl₂.

According to some embodiments, the method for profiling methylation changes in a DNA sample as described herein comprising: profiling methylation of the DNA sample using HinP1I and AciI digestion; and comparing the methylation profile to one or more reference methylation profile. In some embodiments, the DNA sample is cell-free DNA extracted from a biological fluid.

According to some embodiments, the method for profiling methylation of a DNA sample comprising: subjecting the DNA sample to digestion with the methylation-sensitive restriction endonucleases HinP1I and AciI, thereby obtaining restriction endonuclease-treated DNA comprising restriction endonuclease-generated DNA fragments; adding a chelating agent and amplifying at least one restriction locus.

Digestion efficacy can be evaluated either internally to the examined sample, or externally. Internal evaluation can be performed by measuring intact cut sites of genomic positions that are known to be ubiquitously unmethylated. An example of such a locus can be any site on the mitochondrion DNA. External evaluation of digestion efficacy can be performed either by including an unmethylated sample in the digestion step, digesting both samples in parallel, and then verifying that the unmethylated sample was indeed digested (by measuring numbers of intact cut sites). Such an unmethylated sample could be, for example, PCR amplicons, plasmid DNA, commercial unmethylated DNA species, or cell line DNA that is known to be unmethylated in certain genomic positions. Alternatively, external evaluation of digestion efficacy can be achieved in a single step, by spiking in an unmethylated sample into the interrogated sample, and measuring the digestion of the unmethylated DNA sample in the same step as the interrogated sample. For this purpose, it is possible to use all types of unmethylated DNA species mentioned above. In some embodiments, the use of small targets is preferred, such as PCR amplicons or plasmid DNA.

According to some embodiments, DNA digestion may be carried out to complete digestion. In some exemplary embodiments, the methylation-sensitive restriction endonuclease is HinP1I and/or AciI, and complete digestion may be achieved following one to two hours incubation with the enzyme(s) at 37° C. According to certain embodiments, the complete digestion is achieved following two hours incubation. According to certain embodiments, the complete digestion may be achieved following 3, 4, 5, 6, 7, 8, 9, or 10 hours. Each possibility represents a separate embodiment of the invention. The incubation time sufficient for complete digestion is varied and depends on a number of factors, such as the type of restriction enzyme, sample purity, amount of DNA, and DNA integrity. One hour of incubation may be inadequate under certain circumstances, and routine tests may be applied in order to confirm complete digestion.

### DNA Amplification

According to some embodiments, amplification of the genomic loci may be carried out using real-time PCR (RT-PCR), also known as quantitative PCR (qPCR), in which simultaneous amplification and detection of the amplification products are performed.

According to some embodiments, detection of the amplification products in RT-PCR may be achieved using polynucleotide probes, typically fluorescently-labeled polynucleotide probes.

As used herein, “polynucleotide probes” or “oligonucleotide probes” are interchangeable and refer to labeled polynucleotides which are complementary to specific sub-sequences within the nucleic acid sequences of loci of interest, for example, within the sequence of a restriction locus or a control locus. In some embodiments, detection is achieved by using TaqMan assays based on combined reporter and quencher molecules (Roche Molecular Systems Inc.). In such assays, the polynucleotide probes have a fluorescent moiety (fluorophore) attached to their 5′ end and a quencher attached to the 3′ end. During PCR amplification, the polynucleotide probes selectively hybridize to their target sequences on the template, and as the polymerase replicates the template it also cleaves the polynucleotide probes due to the polymerase's 5′-nuclease activity. When the polynucleotide probes are intact, the close proximity between the quencher and the fluorescent moiety normally results in a low level of background fluorescence. When the polynucleotide probes are cleaved, the quencher is decoupled from the fluorescent moiety, resulting in an increase of intensity of fluorescence. The fluorescent signal correlates with the amount of amplification products, i.e., the signal increases as the amplification products accumulate.

As used herein, “selectively hybridize to” (as well as “selective hybridization,” “specifically hybridize to,” and “specific hybridization”) refers to the binding, duplexing, or hybridizing of a nucleic acid molecule (such as a primer or a probe) preferentially to a particular complementary nucleotide sequence under stringent conditions. The term “stringent conditions” refers to conditions under which a nucleic acid molecule will hybridize preferentially to its target sequence and to a lesser extent to, or not at all to, other non-target sequences. A “stringent hybridization” in the context of nucleic acid hybridization is sequence-dependent, and differs under different conditions, as known in the art.

Polynucleotide probes may vary in length. According to some embodiments, the polynucleotide probes may include between 15-30 bases. In additional embodiments, the polynucleotide probes may include between 25-30 bases. In some embodiments, the polynucleotide probes may include between 20-30 bases, for example, 20 bases, 21 bases, 22 bases, 23 bases, 24 bases, 25 bases, 26 bases, 27 bases, 28 bases, 29 bases, 30 bases. Each possibility represents a separate embodiment of the present invention.

Polynucleotide probes may be designed to bind to either strand of the template. Additional considerations include the Tm of the polynucleotide probes, which should preferably be compatible to that of the primers. Computer software may be used for designing the primers and probes.

As noted above, the methods disclosed herein may involve simultaneous amplification of more than one target sequence (at least one restriction locus and one control locus) in the same reaction mixture. In order to distinguish between multiple target sequences that are amplified in parallel, polynucleotide probes labeled with distinct fluorescent colors may be used.

According to) some embodiments, the polynucleotide probes form fluorophore/quencher pairs as known in the art and include, for example, FAM-TAMRA, FAM-BHQ1, Yakima Yellow-BHQ1, ATTO550-BHQ2 and ROX-BHQ2.

According to some embodiments, the dye combinations may be compatible to the RT-PCR thermocycler of choice.

According to some embodiments, fluorescence may be monitored during each PCR cycle, providing an amplification plot showing the change of fluorescent signals from the probes as a function of cycle number.

In the context of real-time PCR, the following terminology is used:

“Quantification cycle” (“Cq”) refers to the cycle number in which fluorescence increases above a threshold, set automatically by software or manually by the user. In some embodiments, the threshold may be constant for all loci and may be set in advance, prior to carrying out the amplification and detection. In other embodiments, the threshold may be defined separately for each locus after the run, based on the maximum fluorescence level detected for this locus during the amplification cycles.

“Threshold” refers to a value of fluorescence used for Cq determination. In some embodiments, the threshold value may be a value above baseline fluorescence, and/or above background noise, and within the exponential growth phase of the amplification plot.

“Baseline” refers to the initial cycles of PCR where there is little to no change in fluorescence.

Computer software may be used to analyze amplification plots and determine baseline, threshold and Cq.

According to alternative embodiments, amplification and detection of amplification products may be carried out by conventional PCR. According to some embodiments, the PCR is performed using fluorescently-labeled primers followed by capillary electrophoresis of amplification products. According to some embodiments, following amplification the amplification products are separated by capillary electrophoresis and fluorescent signals are quantified. In some embodiments, an electropherogram plotting the change in fluorescent signals as a function of size (bp) or time from injection may be generated, wherein each peak in the electropherogram corresponds to the amplification product of a single locus. The peak's height (provided for example using “relative fluorescent units”, rFU) may represent the intensity of the signal from the amplified locus. Computer software may be used to detect peaks and calculate the fluorescence intensities (peak height) of a set of loci whose amplification products were run on the capillary electrophoresis machine, and subsequently the ratios between the signal intensities.

For DNA samples digested with a methylation-sensitive restriction enzyme, e.g., HinP1I, loci in which the CG dinucleotide in the enzyme's recognition site is methylated produce a relatively strong signal (higher peak) in the electropherogram. Conversely, loci in which the CG dinucleotide in the enzyme's recognition site is unmethylated produce a relatively weak signal (lower peak) in the electropherogram.

According to some embodiments, the fluorescent labels of the primers include any one of fluorescein, FAM, lissamine, phycoerythrin, rhodamine, Cy2, Cy3, Cy3.5, Cy5, Cy5.5, Cy7, FluorX, JOE, HEX, NED, VIC and ROX.

### Library Preparation and Sequencing

According to some embodiments, the methods described herein comprise a step of preparing a sequencing library.

According to some embodiments, preparing a sequencing library comprises introducing adapter sequences into DNA fragments in a restriction endonuclease-treated DNA using PCR amplification. In some embodiments, the adapter sequences are introduced using a 2-step PCR, wherein the first PCR is carried out using primers that contain locus-specific sequences and overhang sequences that introduce a first portion of the adapter sequences, and the second PCR is carried out using primers that introduce a second portion of the adapter sequences and optionally sample indices and/or barcodes.

In some embodiments, an NGS sequencing library is generated by: (i) co-amplifying from a restriction endonuclease-treated DNA at least one restriction locus differentially methylated between normal and disease DNA and at least one control locus in a single reaction mixture using locus-specific primers comprising 5′ overhang sequences that introduce a first portion of adapter sequences; (ii) purifying the amplification products; and (iii) conducting a second PCR that introduces a second portion of adapter sequences and optionally sample indices and/or barcodes.

According to additional embodiments, preparing a sequencing library is carried out following locus-specific amplification. In some embodiments, preparing a sequencing library comprises ligating sequencing adapters to amplification products.

According to some embodiments, the step of preparing a sequencing library is carried out on the digested DNA without a step of DNA amplification. In other embodiments, the step of preparing a sequencing library is carried out after a step of DNA amplification.

According to some embodiments, library preparation for sequencing according to the present invention is carried out in an end-preserving manner, indicating that the library preparation process does not include PCR to enrich genomic regions of interest and/or introduce sequencing adapters. According to these embodiments, library preparation comprises adding sequencing adapters via ligation (e.g., enzymatic ligation). If enrichment of certain genomic regions is desired, library preparation according to these embodiments comprises enriching the genomic regions of interest using capture agents.

According to some embodiments, the present invention relates to compositions and methods for high resolution DNA methylation profiling. In some embodiments, the present invention provides the use of methylation-sensitive/methylation-dependent restriction enzymes and high-throughput sequencing in the analysis of DNA methylation. In some particular embodiments, the present invention provides the use of methylation-sensitive/methylation-dependent restriction enzymes and high-throughput sequencing for direct calculation of methylated and unmethylated DNA levels.

Methylation in the human genome occurs in the form of 5-methyl cytosine and is confined to cytosine residues that are part of the sequence CG, also denoted as CpG dinucleotides (cytosine residues that are part of other sequences are not methylated). Some CG dinucleotides in the human genome are methylated, and others are not. In addition, methylation is cell and tissue specific, such that a specific CG dinucleotide can be methylated in a certain cell and at the same time unmethylated in a different cell, or methylated in a certain tissue and at the same time unmethylated in different tissues. DNA methylation is an important regulator of gene transcription.

The methylation pattern of cancer DNA differs from that of normal DNA, wherein some loci are hypermethylated while others are hypomethylated. In some embodiments, the present invention provides methods and compositions for sensitive detection of differentially methylated (e.g., hypermethylated) genomic loci associated with cancer.

According to an aspect, the present invention provides a method for processing a cell-free DNA sample to obtain sequencing data for genetic and epigenetic analysis, the method comprising:
(i) subjecting the cell-free DNA sample to digestion with at least one methylation-sensitive restriction endonuclease, to obtain restriction endonuclease-treated DNA in which methylated restriction sites are intact and unmethylated restriction sites are cut;
(ii) adding a chelating agent; and
(iii) preparing a sequencing library from the restriction endonuclease-treated DNA while preserving the sequence information at the ends of the DNA molecules, wherein preparing the sequencing library comprises ligating sequencing adapters to DNA molecules in the restriction endonuclease-treated DNA, wherein each adapter is capable of ligation to both the digested and undigested DNA molecules.

According to an aspect, the present invention provides a method for profiling genetic and epigenetic characteristics of a cell-free DNA (cfDNA) sample from a subject, the method comprising:
(i) subjecting the cell-free DNA sample to digestion with at least one methylation-sensitive restriction endonuclease, to obtain restriction endonuclease-treated DNA in which methylated sites are intact and unmethylated sites are cut;
(ii) adding a chelating agent;
(iii) preparing a sequencing library from the restriction endonuclease-treated DNA while preserving the sequence information at the ends of the DNA molecules, wherein the preparation of a sequencing library is carried out in a buffer comprising 0.5-5 mM of a chelating agent, wherein preparing the sequencing library comprises ligating sequencing adapters to DNA molecules in the restriction endonuclease-treated DNA, wherein each adapter is capable of ligation to both the digested and undigested DNA molecules;
(iv) sequencing the sequencing library by a high-throughput sequencing method to provide sequencing data; and
(v) determining from the sequencing data a methylation value for at least one restriction locus and optionally at least one additional genetic or epigenetic characteristic of the cell-free DNA sample selected from DNA mutation, copy number variation and nucleosome positioning.

According to some embodiments, the amount of cell-free DNA comprising no more than 3,000 haploid equivalents is sufficient for the method, wherein the cell-free DNA sample is not subjected to amplification prior to library preparation, and wherein determining the methylation value and the at least one additional genetic or epigenetic characteristic of the cell-free DNA sample is carried out based on the same sequencing data.

As used herein, 3.3 pg of DNA corresponds to 1 haploid equivalent.

According to some embodiments, 10 ng of DNA are sufficient for the methods disclosed herein. In additional embodiments, 20 ng of DNA are sufficient for the methods disclosed herein. In additional embodiments, the methods disclosed herein are carried out using an initial amount of DNA ranging from 10-200 ng, for example between 20-200 ng, between 20-100 ng, including each value within the ranges. Each possibility represents a separate embodiment.

According to some embodiments, 3,000 haploid equivalents are sufficient for the methods disclosed herein. In additional embodiments, 6,000 haploid equivalents are sufficient for the methods disclosed herein. In additional embodiments, the methods disclosed herein are carried out using an initial amount of DNA comprising 3,000-60,000 haploid equivalents, for example between 6,000-60,000 haploid equivalents, between 6,000-30,000 haploid equivalents, including each value within the ranges. Each possibility represents a separate embodiment.

According to some embodiments, there is provided herein a method for profiling methylation of a DNA sample from a subject, the method comprising:
(i) subjecting the DNA sample to digestion with at least one methylation-sensitive restriction endonuclease, to obtain restriction endonuclease-treated DNA in which methylated sites are intact and unmethylated sites are cut;
(ii) adding a chelating agent;
(iii) preparing a sequencing library from the restriction endonuclease-treated DNA;
(iv) sequencing the sequencing library by a high-throughput sequencing method to obtain sequence reads;
(v) selecting at least one restriction locus and determining the number of sequence reads covering a predefined genomic region of at least 50 bps in length that contains said restriction locus; and
(vi) determining a methylation value for the at least one restriction locus based on the read count determined in step (iv) and a reference read count,
thereby profiling methylation of the cell-free DNA sample.

According to some embodiments, profiling methylation of a DNA sample comprises determining the number of sequence reads covering a predefined genomic region of at least 60 bps in length that contains said restriction locus, for example a predefined genomic region of at least 70 bps, at least 80 bps, at least 90 bps, at least 100 bps, between 50-150 bps, between 50-120 bps, between 50-100 bps that contains the restriction locus. Each possibility represents a separate embodiment.

According to some embodiments, the at least one restriction locus is located within a CG-island. “CG islands” (or CpG islands) are regions of DNA with a high G/C content and a high frequency of CG dinucleotides relative to the whole genome of an organism of interest. CG islands are typically between 200-3,000 bps in length and are typically characterized by a GC content greater than 50% and an observed: expected CG ratio of more than 0.6. Genomic regions of lower CG density are termed “CG oceans” and comprise most of the genome.

According to some embodiments, there is provided a method for identifying the presence or absence of a disease in a subject, comprising: profiling methylation of a DNA sample from the subject as disclosed herein; comparing the methylation profile of the DNA sample to one or more reference methylation profile; and determining the presence or absence of the disease in the subject based on the comparison.

According to some embodiments, there is provided a method for identifying a DNA methylation marker indicative of the source of a DNA sample comprising profiling methylation as disclosed herein. In additional embodiments, there is provided herein a method for assessing the quality of a DNA methylation marker comprising profiling methylation as disclosed herein. In some embodiments, the DNA methylation marker is a marker indicative of the presence or absence of a disease, e.g., a type of cancer. In additional embodiments, the DNA methylation marker is a marker indicative of a stage of a disease, e.g., a cancer stage. In additional embodiments, the DNA methylation marker is a marker indicative of a type of tissue (e.g., lung tissue, breast tissue, colon tissue etc.).

According to some embodiments, there is provided the use of sequence reads produced following digestion of a DNA sample with at least one methylation-sensitive restriction enzyme and/or at least one methylation-dependent restriction enzyme and high-throughput sequencing, for profiling methylation of the DNA sample by direct determination of methylated and unmethylated DNA levels of at least one restriction locus in the DNA sample, wherein said determination of methylated and unmethylated DNA levels is based on the same sequencing data.

In general, embodiments which can be performed with methylation-sensitive restriction enzyme(s) can be done alternatively with methylation-dependent restriction enzyme(s), and downstream steps will be adjusted accordingly. For example, in some embodiments, following high-throughput sequencing and generation of sequence reads, a method for profiling methylation according to the present invention comprises: selecting at least one restriction locus and determining the number of sequence reads covering a predefined genomic region of at least 50 bps in length that contains said restriction locus; and calculating a methylation value based on the read count of the predefined genomic region and a reference read count, the calculated methylation value reflects the number of molecules that were unmethylated in the DNA sample and therefore remained intact following digestion with methylation-dependent restrictions enzymes(s).

As another example, in some embodiments, for calculating a level of methylated DNA of a restriction locus, following high-throughput sequencing and generation of sequence reads, the method comprises: determining from the sequence reads a read count of sequence reads starting or ending at a nucleotide within the restriction locus, the read count representing the number of DNA molecules in the DNA sample in which said restriction locus was methylated and therefore cut by the restriction endonuclease; and calculating a level of methylated DNA at the restriction locus based on the determined read count of sequence reads starting or ending at a nucleotide within the restriction locus. For calculating a level of unmethylated DNA of a restriction locus, in some embodiments, the method comprises: determining from the sequence reads a read count of the restriction locus, the read count representing the number of DNA molecules in the DNA sample in which said restriction locus was unmethylated and therefore remained intact; and calculating a level of unmethylated DNA at the restriction locus based on the determined read count of the restriction locus.

“High throughput sequencing,” (also termed “next generation sequencing”) includes sequence determination using methods that determine many (typically thousands to billions) of nucleic acid sequences in parallel. High throughput sequencing generally involves three basic steps: library preparation, sequencing and data analysis. Examples of high throughput sequencing techniques include sequencing-by-synthesis and sequencing-by-ligation (employed, for example, by Illumina Inc., Life Technologies Inc., Roche), nanopore sequencing methods and electronic detection-based methods such as Ion Torrent™ technology (Life Technologies Inc.).

Library preparation for the major high-throughput sequencing platforms requires the addition (typically ligation or via PCR) of specific adapter oligonucleotides to fragments of the DNA to be sequenced. As disclosed herein, restriction digestion is preferably carried out before adapter ligation to avoid possible digestion of the adapters by the enzymes.

The digestion of DNA by the methylation-sensitive/dependent restriction endonuclease(s) as disclosed herein typically does not result in homogeneous, blunt-ended fragments. Thus, in some embodiments, end repair is needed to ensure that each DNA molecule is free of overhangs, and contains 5′ phosphate and 3′ hydroxyl groups. A typical blunting enzyme mix includes a polymerase and a polynucleotide kinase, for example, T4 DNA polymerase and T4 polynucleotide kinase (PNK). T4 DNA polymerase (in the presence of dNTPs) can fill-in 5′ overhangs and trim 3′ overhangs down to the dsDNA interface to generate the blunt ends. The T4 PNK can then phosphorylate the 5′ terminal nucleotide. For Illumina libraries, incorporation of a non-templated deoxyadenosine 5′-monophosphate (dAMP) onto the 3′ end of blunted DNA fragments, a process known as dA-tailing, is also required for library preparation. dA-tails prevent concatamer formation during downstream ligation steps, and enable DNA fragments to be ligated to adapter oligonucleotides with complementary dT-overhangs.

Adapter oligonucleotides are also termed herein “sequencing adapters” or “adapter sequences”. In some embodiments, sequencing adapters are ligated to the DNA fragments using end-preserving methods such as enzymatic ligation in which a ligase enzyme covalently links a sequencing adapter to a DNA fragment, making a complete library molecule. In other embodiments, sequencing adapters are introduced via PCR. Sequencing adapters are added (e.g. ligated or via PCR) at the 5′ and 3′ ends of DNA fragments in the sequencing library. Sequencing adapters typically include platform-specific sequences for fragment recognition by a particular sequencer: for example, sequences that enable library fragments to bind to the flow cells of Illumina platforms. Each sequencing instrument provider typically uses a specific set of sequences for this purpose.

Sequencing adapters may also include sample indices. “Sample indices”, also termed “sample barcodes” are sequences that enable multiple samples to be sequenced together (i.e., multiplexed) on the same instrument flow cell or chip. Each sample index, typically 6-10 bases, is specific to a given sample library and is used for de-multiplexing during data analysis to assign individual sequence reads to the correct sample. Sequencing adapters may contain single or dual sample indexes depending on the number of libraries combined and the level of accuracy desired.

Sequencing adapters may include unique molecular identifiers (UMIs). UMIs are a type of molecular barcodes that provide molecular tracking, error correction and increased accuracy during sequencing. UMIs are short sequences, typically 5 to 20 bases in length, used to uniquely tag each molecule in a sample library. Since each nucleic acid in the starting material is tagged with a unique molecular barcode, bioinformatics software can filter out duplicate reads and PCR errors with a high level of accuracy and report unique reads, removing the identified errors before final data analysis.

In some embodiments, both a sample barcode sequence and a UMI are incorporated into a nucleic acid target molecule.

High-throughput sequencing according to the present invention may be performed using various high-throughput sequencing instruments and platforms, including but not limited to: Novaseq™, Nextseq™ and MiSeq™ (Illumina), 454 Sequencing (Roche), Ion Chef™ (ThermoFisher), SOLiD® (ThermoFisher) and Sequel II™ (Pacific Biosciences). The appropriate platform-designed sequencing adapters are used for preparing the sequencing library.

According to some embodiments, whole genome sequencing is performed on libraries prepared from endonuclease-treated DNA. The libraries are prepared using sequencing adapters suitable for the sequencing platform being used.

According to other embodiments, region(s) of interest in the endonuclease-treated DNA can be amplified using specific primers, followed by library preparation and high-throughput sequencing. According to additional embodiments, region(s) of interest in the endonuclease-treated DNA may be captured using, for example, a solution-phase or solid-phase hybridization-based process, followed by the high-throughput sequencing. Enrichment or amplification of regions of interest followed by high-throughput sequencing is referred to herein as “target-specific high-throughput sequencing”. Target-specific high-throughput sequencing includes, for example, CpG island sequencing and exome sequencing. Target-specific high-throughput sequencing also includes sequencing of specific informative genomic regions, for example, regions known to be differentially methylated between cancer and non-cancer tissues. Capture of genomic regions for target-specific sequencing is typically carried out after library preparation. In some embodiments, the methods disclosed herein comprise enriching genomic regions of interest.

According to some embodiments, a method for genetic and epigenetic profiling of DNA samples according to the present invention comprises:
extracting DNA from a biological sample;
subjecting the extracted DNA to digestion with at least one methylation-sensitive restriction endonuclease, thereby obtaining restriction endonuclease-treated DNA;
adding a chelating agent;
preparing a sequencing library from the restriction endonuclease-treated DNA using sequencing adapters ligated to DNA fragments in the restriction endonuclease-treated DNA, said preparing is done in a reaction buffer comprising 0.5 to 5 mM chelating agent;
enriching at least one (preferably a plurality of) genomic regions of interest from the sequencing library using capture agents, to obtain a sequencing library enriched with the at least one (preferably a plurality of) genomic regions of interest;
subjecting the sequencing library enriched with the at least one (preferably a plurality of) genomic regions of interest to high-throughput sequencing; and
determining from the sequencing data a methylation value for at least one restriction locus and optionally at least one additional genetic or epigenetic characteristic of the cell-free DNA sample selected from DNA mutation, copy number variation and nucleosome positioning as disclosed herein.

### Analysis of Sequence Reads

According to some embodiments, the DNA sample for use according to the present invention is a cell-free DNA sample.

According to some embodiments, the methods of the present invention comprise a step of sequencing a library by a high-throughput sequencing method to obtain sequencing data.

According to some embodiments, the preparation of the sequencing library is carried out in a buffer comprising 0.5-5 mM of a chelating agent.

According to some embodiments, the amount of cell-free DNA comprising 3000 haploid equivalents is sufficient to achieve at least one of: unique mapping rate of at least 85%, a copy number integrity characterized by Pearson correlation of at least 0.65 compared to undigested sample and nucleosome positioning integrity characterized by Pearson correlation of at least 0.55 compared to undigested sample.

According to some embodiments, an amount of cell-free DNA comprising 6,000 haploid equivalents is sufficient for the methods disclosed herein.

According to some embodiments, “sequence reads” (or simply, “reads”), namely, nucleotide sequences produced by the sequencing process, are mapped against a reference genome. A “reference genome” as used herein refers to a previously identified genome sequence, whether partial or complete, assembled as a representative example of a species or subject. A reference genome is typically haploid, and typically does not represent the genome of a single individual of the species but rather is a mosaic of the genomes of several individuals. A reference genome for the methods of the present invention is typically a human reference genome. In some embodiments, the reference genome is the complete human genome, such as the human genome assemblies available at the website of the National Center for Biotechnology Information (NCBI) or at the University of California, Santa Cruz (UCSC) Genome Browser. An example of a suitable reference genome for human studies is the ‘hg18’ genome assembly. As an alternative, the more recent GRCh38 major assembly can be used (going up to patch p13).

Read mapping is the process to align the reads on a reference genome in order to identify the location of the reads within the reference genome. The sequence reads that align are designated as being “mapped”. The alignment process aims to maximize the possibility for obtaining regions of sequence identity across the various sequences in the alignment, allowing mismatches, indels and/or clipping of some short fragments on the two ends of the reads. The number of reads mapped to a certain genomic locus of interest is referred to herein as the “read count” or “copy number” of this genomic locus. Computer software may be used to analyze sequence reads, map sequence reads against a reference genome and quantify the number of reads.

The terms “genomic locus” and “locus” as used herein are interchangeable and refer to a DNA sequence at a specific location within the genome. A “locus” may include a single position (a single nucleotide at a defined position in the genome) or a stretch or nucleotides starting and ending at defined positions in the genome. The specific position(s) may be identified by the molecular location, namely, by the chromosome and the numbers of the starting and ending base pairs on the chromosome. A variant of a DNA sequence at a given genomic position is called an allele. Alleles of a locus are located at identical sites on homologous chromosomes. Genomic loci include gene sequences as well as other genetic elements (e.g., intergenic sequences).

A “restriction locus” is used herein to describe a genomic locus which is a restriction site of a methylation-sensitive/-dependent restriction endonuclease applied in the digestion step according to the present invention. Restriction loci according to the present invention may be differentially methylated between normal and disease DNA, meaning that for a given disease for which the analysis is carried out, for example, a certain type of cancer, the restriction loci differ in their methylation level between normal DNA and DNA derived from cancer cells. For example, DNA from the cancer cells may have an increased methylation level at the restriction loci compared to normal non-cancerous DNA. More particularly, the restriction loci contain CG dinucleotides that are more methylated in cancer DNA compared to normal non-cancerous DNA. According to the present invention, the differentially methylated CG dinucleotides are located within recognition sites of the at least one restriction enzyme applied in the digestion step.

According to some embodiments, a restriction locus according to the present invention contains a CG dinucleotide which is more methylated in cell-free DNA, e.g., plasma DNA, of subjects with a certain type of cancer than in cell-free DNA of healthy subjects. In some embodiments, plasma samples of the cancer patients contain a greater proportion of DNA molecules that are methylated at the restriction locus compared to plasma samples of healthy subjects.

According to additional embodiments, a restriction locus according to the present invention contains a CG dinucleotide which is more methylated in DNA from a cancerous tissue (e.g., a tumor sample) than in DNA from a non-cancerous tissue, meaning that in the cancerous tissue a greater proportion of DNA molecules are methylated at this position compared to the non-cancerous tissue.

A methylation-sensitive restriction enzyme cleaves its recognition sequence only if it is unmethylated. A methylation-dependent restriction enzyme cleaves its recognition sequence only if it is methylated. Thus, differences in methylation levels between samples result in differences in the degree of digestion, and subsequently different amounts of sequence reads in the following sequencing and quantification steps. Such differences enable distinguishing between DNA from different samples, for example, between DNA samples from subjects with cancer and DNA samples from healthy subjects.

The terms “level of methylated DNA”, “methylation level” or “methylation value” of a restriction locus is a numerical value representing the number of DNA molecules that are methylated at this restriction locus (namely, methylated at a CG dinucleotide within the restriction locus) out of the total number of DNA molecules containing the restriction locus in the sample. In some embodiments, the level of methylated DNA of a restriction locus is calculated herein from the read count of the restriction locus following digestion with at least one methylation-sensitive restriction endonuclease. In additional embodiments, the level of methylated DNA of a restriction locus is calculated herein from the read count of a predefined genomic region of at least 50 bps that contains the restriction locus. As methylation-sensitive restriction endonucleases cleave their recognition sequence only if it is unmethylated, the read count of the restriction locus represents the number of DNA molecules in the DNA sample in which the restriction locus was methylated and therefore remained intact.

According to some embodiments, the methylation level of the restriction locus is calculated by dividing the read count of the restriction locus, or the read count of a predefined genomic region of at least 50 bps that contains the restriction locus, by an expected read count of the restriction locus or the predefined genomic region of at least 50 bps that contains the restriction locus. An expected read count of the restriction locus/predefined genomic region may be determined, for example, using: (i) read count of a reference locus/genomic region of the same length as the restriction locus/genomic region, that is not cut by the restriction endonuclease; (ii) average read count of a plurality of reference loci/genomic regions of the same length as the restriction locus/genomic region, that are not cut by the restriction endonuclease; or (iii) read count of the restriction locus/predefined genomic region in an undigested control DNA sample, optionally corrected for sequencing depth differences.

The terms “level of unmethylated DNA” or “unmethylation level” of a restriction locus is a numerical value representing the number of DNA molecules that are unmethylated at this restriction locus (namely, unmethylated at a CG dinucleotide within the restriction locus) out of the total number of DNA molecules containing the restriction locus in the sample. In some embodiments, the level of unmethylated DNA of a restriction locus is calculated from the number of reads starting or ending at a nucleotide within the restriction locus following digestion with at least one methylation-sensitive restriction endonuclease and any subsequent end repair. The exact nucleotide within the restriction locus in which the sequence reads start or end depends on the type of restriction endonuclease used in the digestion step and the length of its recognition sequence. For example, for restriction endonucleases that produce non-blunt ends with 5′ overhangs, digestion and end repair result in fragments that start at the second nucleotide of the recognition sequence and fragments that end at the penultimate nucleotide of the recognition sequence.

As methylation-sensitive restriction endonucleases cleave their recognition sequence only if it is unmethylated, the number of reads starting or ending at a nucleotide within the restriction locus represent the number of DNA molecules in the DNA sample in which the restriction locus was unmethylated and therefore cut by the restriction endonuclease.

Thus, in some embodiments, the method of the present invention comprises: determining a number of sequence reads starting at a nucleotide within the restriction locus; determining a number of sequence reads ending at a nucleotide within the restriction locus; and calculating a level of unmethylated DNA at the restriction locus using the orientation that provides the larger number of sequence reads. In additional embodiments, the method of the present invention comprises: determining a number of sequence reads starting at a nucleotide within the restriction locus; determining a number of sequence reads ending at a nucleotide within the restriction locus; calculating an average between the two values; and using the average to calculate a level of unmethylated DNA at the restriction locus.

According to additional embodiments, the level of unmethylated DNA is calculated by determining a total fragment number, which is determined from the read count of the restriction locus and read count of sequence reads starting or ending at a nucleotide within the restriction locus.

According to some embodiments, the level of unmethylated DNA is expressed as percentage (%) of unmethylation, representing the percentage of DNA molecules that are unmethylated at the restriction locus out of the total number of DNA molecules containing the restriction locus in the sample.

According to an aspect, the present invention provides a method for processing a DNA sample to obtain sequencing data for genetic and epigenetic analysis, the method comprising:
(i) subjecting the DNA sample to digestion with at least one methylation-sensitive restriction endonuclease, to obtain restriction endonuclease-treated DNA in which methylated restriction sites are intact and unmethylated restriction sites are cut;
(ii) adding a chelating agent; and
(iii) preparing a sequencing library from the restriction endonuclease-treated DNA while preserving the sequence information at the ends of the DNA molecules, wherein preparing the sequencing library comprises ligating sequencing adapters to DNA molecules in the restriction endonuclease-treated DNA, wherein each adapter is capable of ligation to both the digested and undigested DNA molecules.

### Detecting Methylation Changes

As used herein, “detecting methylation changes” refers to detecting whether a tested DNA sample contains methylation changes compared to one or more reference DNA samples, detecting whether a DNA sample is characterized by a different methylation profile at selected genomic loci compared to a reference methylation profile, and/or determining whether the methylation profile of a DNA sample is normal or contains methylation changes indicative of the presence of a disease. Each possibility represents a separate embodiment of the present invention. Detecting methylation changes also encompasses comparing methylation data obtained as disclosed herein between samples in order to identify genomic regions differentially methylated between the samples, which may be used as DNA methylation markers. For example, methylation data obtained as disclosed herein may be analyzed to identify genomic regions differentially methylated between different types of tissues, between cancer and non-cancer DNA, between different types of cancer, or between different stages of a certain type of cancer. In some embodiments, the methods disclosed herein provide genome-wide methylation analysis. In other embodiments, the methods disclosed herein provide target-specific methylation analysis. Computer software may be used in the analysis of the sequencing and methylation data.

The methods of the present invention may be applied for identifying and analyzing DNA methylation marker regions which may be used as cancer diagnostic markers. According to some embodiments, markers are of a cancer selected from the group consisting of lung cancer, colorectal cancer, liver cancer, breast cancer, pancreatic cancer, uterine cancer, ovarian cancer, head & neck cancer, gastric cancer, esophageal cancer, hematological cancers (e.g. lymphoma) and sarcoma. In some embodiments, the marker are used as pan-cancer markers. The methods may also be applied for identifying differential methylation between different types of cancer, for example, determining methylation profiles characteristic of different types of cancer, that can differentiate between different types of cancer. The methods disclosed herein are applicable to any type of cancer, including, but not limited to: lung cancer, bladder cancer, breast cancer, colorectal cancer, prostate cancer, gastric cancer, skin cancer (e.g. melanoma), cancer affecting the nervous system, bone cancer, ovarian cancer, liver cancer (e.g. hepatocellular carcinoma), hematologic malignancies, pancreatic cancer, kidney cancer, cervical cancer. Each type of cancer is a separate embodiment of the present invention. The methods of the present invention may also be applied to identify tissue-specific methylation markers. For example, to identify methylation markers specific for: lung, bladder, breast, colorectal, prostate, gastric, ovarian, pancreas, kidney, cervical tissue. Each type of tissue is a separate embodiment of the present invention. Such markers may be used, for example, to identify the tissue source of circulating cell-free DNA.

The methods of the present invention may be applied for identifying a disease (e.g., a cancer) in a subject. “Identifying a disease” as used herein encompasses any one or more of screening for the disease, detecting the presence or absence of the disease, detecting recurrence of the disease, detecting susceptibility to the disease, detecting response to treatment, determining efficacy of treatment, determining stage (severity) of the disease, determining prognosis and early diagnosis of the disease in a subject. Each possibility represents a separate embodiment of the present invention.

“Assessing cancer” or “assessing the presence of cancer” or “assessing the presence or absence of cancer” as used herein refer to determining the likelihood that a subject has cancer. The terms encompass determining whether a subject should be subjected to confirmatory cancer testing to confirm (or rule out) the presence of cancer, such as confirmatory blood tests, urine tests, cytology, imaging, endoscopy and/or biopsy. The terms further encompass aiding the diagnosis of cancer in a subject. The terms further encompass quantifying cancer-related changes in cell-free DNA samples which are indicative for the presence of cancer. Assessing the presence of cancer according to the present invention includes one or more of screening for cancer, assessing recurrence of cancer, assessing susceptibility or risk to cancer, assessing and/or monitoring response to treatment, assessing efficacy of treatment, assessing severity (stage) of cancer and assessing prognosis of cancer in a subject. Each possibility represents a separate embodiment of the present invention. It is to be understood that a negative result in the assays disclosed herein is still considered an assessment for the presence of cancer according to the present invention.

The methods of the present invention may further include a step of determining a tumor fraction, or fractional concentration of tumor DNA. Tumor fraction is the proportion of tumor molecules in a cfDNA sample.

Determining a “methylation profile” (or “DNA methylation profile” or “methylation profile of a DNA sample”) as disclosed herein refers to determining methylation values at one or more restriction loci, preferably at a plurality of restriction loci. In some embodiments, determining a methylation profile comprises determining levels of methylated and unmethylated DNA at one or more restriction loci, preferably at a plurality of restriction loci.

A “reference methylation profile” as disclosed herein refers to a methylation profile determined in DNA from a known source. A “reference DNA sample” is a DNA sample from a known source. In some embodiments, a reference methylation profile is a profile determined in a plurality of reference DNA samples. In addition, the methods of the present invention may be used for analyzing (e.g., measuring) methylation changes between DNA samples taken from a single subject at different time points, for example, taken at different stages of a disease, or taken before and after treatment of a disease. The methylation profile of the DNA sample taken at a first time point may be used as a reference for the methylation profile of a DNA sample taken at a second (later) time point.

A “reference methylation level” for a particular restriction locus or a particular genomic region spanning a plurality of restriction loci is the level of methylation measured for the particular restriction locus/genomic region in DNA from a known source. A “reference methylation value” for a particular restriction locus or a particular genomic region spanning a plurality of restriction loci is a numerical value representing the level of methylation of the particular restriction locus/genomic region in DNA from a known source.

A “reference level of unmethylated DNA” for a particular restriction locus or a particular genomic region spanning a plurality of restriction loci is the level of unmethylated DNA measured for the particular restriction locus/genomic region in DNA from a known source.

According to some embodiments, the methods disclosed herein are diagnostic methods. According to some embodiments, diagnostic methods disclosed herein comprise pre-determination of reference methylation and/or unmethylation from disease DNA. In some embodiments, diagnostic methods of the present invention comprise pre-determination of reference methylation and/or unmethylation from normal DNA as disclosed herein.

Tissue-specific methylation profile can also be characterized using the methods disclosed herein, in order to establish normal non-cancer DNA methylation profile of the tissue. Alternatively or additionally, tissue-specific methylation profile can be characterized in order to identify the tissue source of circulating cell-free DNA.

According to some embodiments, detecting methylation changes according to the present invention comprises identifying the presence or absence of a certain disease in a subject, based on the methylation profile of a DNA sample from the subject.

According to some embodiments, a method for identifying the cell source or tissue source of a DNA sample is provided (e.g., identifying what is the type of tissue from which the DNA is derived, and/or identifying whether the DNA is derived from normal or diseased cells/tissue).

A person of skill in the art would appreciate that the comparison of DNA methylation values and/or unmethylation values calculated for a tested sample to one or more corresponding reference values may be performed in a number of ways, using various statistical means.

According to some embodiments, the methods disclosed herein comprise comparing a plurality of values calculated for a plurality of restriction loci to their corresponding healthy and/or disease references values. In some embodiments, a pattern of values is analyzed using statistical means and computerized algorithm to determine if it represents a pattern of a disease in question or a normal, healthy pattern. Exemplary algorithms include, but are not limited to, machine learning and pattern recognition algorithms.

### Additional Genetic and Epigenetic Characterization

In addition to DNA methylation/unmethylation values, it is possible to obtain from the same sequencing data disclosed herein information on DNA mutations, copy number changes, and nucleosome positioning for cell-free DNA. Generally, cell-free DNA circulates in fragments ranging between 120-220 bp. This pattern agrees with the length of DNA wrapped around a single nucleosome, plus a short stretch of ˜20 bp (linker DNA) bound to a histone. As nucleosome positioning varies between different tissues, and in malignant cells, the pattern of fragmentation has been shown to aid in determining the predominant cell-type of origin contributing to the cfDNA pool.

Advantageously, determination of DNA methylation profile and determination of at least one additional genetic or epigenetic characteristic as disclosed herein may be carried out based on the same sequencing data.

According to some embodiments, a sequencing-based assay as disclosed herein combines detection of methylation changes with mutation detection and analysis of additional epigenetic characteristics, all in one single assay. The assay advantageously allows combined analysis of small amounts of DNA in a single assay.

The combined analysis of methylation and additional genetic and epigenetic characteristics is useful in enhancing detection of cancer (or any other condition/tissue source).

According to some exemplary embodiments, a method for detecting the presence or absence of a cancer in a subject comprises:
(a) profiling methylation of the DNA sample as disclosed herein, to detect the presence or absence of hypermethylation at one or more cancer-associated genomic region; and
(b) one or more of:
determining the presence or absence of one or more cancer-associated mutation (e.g., cancer-associated mutation in oncogenes/tumor suppressors);
determining the presence or absence of cancer-associated copy number variation; and
determining the presence or absence of cancer-associated nucleosomal positioning,
wherein (a) and (b) are carried out using the same sequencing data, and
wherein determining the presence of hypermethylation at one or more cancer-associated genomic region and at least one of: one or more cancer-associated mutation, cancer-associated copy number variation and cancer-associated nucleosomal positioning is indicative of the presence of cancer in the subject.

The non-methylation cancer-associated changes may be combined with methylation information in a dependent or independent manner, depending on whether or not the cancer-associated changes are found on the same DNA fragment, where changes that are found on the same fragment provide a stronger indication for the presence of cancer.

According to some embodiments, there is provided a method for profiling genetic and epigenetic characteristics of a DNA sample, the method comprising: profiling methylation of the DNA sample as disclosed herein; and determining at least one additional genetic or epigenetic characteristic of the DNA sample, wherein the at least one additional genetic or epigenetic characteristic is selected from DNA mutation, copy number variation and nucleosome positioning, wherein profiling the methylation and determining the at least one additional genetic or epigenetic characteristic are carried out using the same sequencing data, thereby profiling genetic and epigenetic characteristics of the DNA sample.

In some embodiments, there is provided a method for detecting the presence or absence of a disease in a subject, the method comprising: profiling methylation of the DNA sample as disclosed herein; and determining at least one additional genetic or epigenetic characteristic of the DNA sample, wherein the at least one additional genetic or epigenetic characteristic is selected from DNA mutation, copy number variation and nucleosome positioning.

### Kits and Reaction Mixtures

According to some embodiments, there is provided herein kits for analyzing a DNA sample. According to some embodiments, there is provided herein kits for detecting methylation changes in a DNA sample. In some embodiments, there is provided herein kits, reaction mixtures and methods for detecting genetic and epigenetic changes in a DNA sample. In additional embodiments, there is provided herein kits for detecting genetic and epigenetic changes in a DNA sample.

According to some embodiments, the kits and reaction mixtures described herein are for profiling methylation of DNA samples according to the methods disclosed herein. In some embodiments, the kits and reaction mixtures are for profiling genetic and epigenetic characteristics of DNA samples according to the methods disclosed herein. In additional embodiments, the kits and reaction mixtures are for detecting genetic and epigenetic changes in a DNA sample according to the methods disclosed herein.

According to some embodiments, a kit or a reaction mix according to the present invention comprises components needed for DNA digestion in addition to the restriction enzyme(s), such as one or more buffers.

According to some embodiments, a kit or a reaction mix according to the present invention comprises components needed for DNA amplification.

According to an aspect, the present invention provides a kit or PCR reaction mix comprising between 0.5 mM and 50 mM chelating agent and a Taq polymerase.

According to an aspect, the present invention provides a kit or PCR reaction mix comprising between 0.5 mM and 10 mM chelating agent and a Taq polymerase.

According to some embodiments, the PCR reaction mix comprises dNTPs.

According to some embodiments, the PCR reaction mix comprising at least one primer. According to some embodiments, the PCR reaction mix comprising a plurality of primers. According to certain embodiments, the PCR reaction mix comprises probes.

According to some embodiments, the PCR reaction mix further comprises restriction enzyme digested DNA.

According to some embodiments, the chelating agent is EDTA. According to other embodiments, the chelating agent is EGTA.

According to some embodiments, the kit or PCR reaction mix comprising between 1-50, 5-50, 10-50, 1-40, 5-40, 10-40, 1-30, 5-30, 10-30 or 10-30 mM of the chelating agent. According to some embodiments, the kit or PCR reaction mix comprising between 2-4, 4-6, 5-10, or 8-10 mM of the chelating agent. Each possibility represents a separate embodiment of the invention.

According to another aspect, the present invention provides a kit comprising an amplification reaction mix comprising between 1 mM and 50 mM chelating agent and a Taq polymerase.

According to some embodiments, the kit comprises at least one methylation-sensitive restriction endonuclease as described hereinabove. According to some embodiments, the kit comprises a digestion buffer comprising magnesium.

As used herein, the term “about”, when referring to a measurable value is meant to encompass variations of +/−10%, more preferably +/−5%, even more preferably +/−1%, and still more preferably +/−0.1% from the specified value.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1—Addition of EDTA Enables Performing PCR After Digestion Without the Need for a Purification Step

DNA digestion was performed in CutSmart buffer (NEB) which contains 10 mM Mg. After digestion, a PCR was performed directly by diluting the digested DNA so the final Mg concentration will match the required concentration of the polymerase (the polymerase buffer does not contain Mg). In the case of amplification based NGS, a high-fidelity polymerase is used and which requires 2.5 mM Mg (KAPA HiFi polymerase—Roche). The magnesium is supplied in the polymerase buffer so the only way to avoid excess Mg is to clean the sample.

In order to avoid DNA loss in the purification step after digestion, it was tested whether the addition of the chelating agent EDTA can be used to reduce the available Mg concentration and enable efficient PCR reaction.

In the following experiment two cfDNA samples were used:
1. cfDNA in AVE buffer (Qiagen)
2. cfDNA with CutSmart buffer X1 (10 mM Mg)

PCR reaction using 2× KAPA HiFi ready mix (Roche) was performed according to the manufacturer instructions on the following DNA samples:

$$1. 2 ng DNA - sample number 1$$

$$2. 2 ng DNA - sample number 2 + 10 mM EDTA$$

$$3. 2 ng DNA - sample number 2 + 5 mM EDTA$$

$$4. 2 ng DNA - sample number 2 + 4 mM EDTA$$

$$5. 2 ng DNA - sample number 2 + 3 mM EDTA$$

$$6. 2 ng DNA - sample number 2 + 2 mM EDTA$$

$$7. 2 ng DNA - sample number 2 + 1 mM EDTA$$

$$8. 2 ng DNA - sample number 2 + 0.5 mM EDTA$$

$$9. 2 ng DNA - sample number 2$$

$$10. DDW - negative control$$

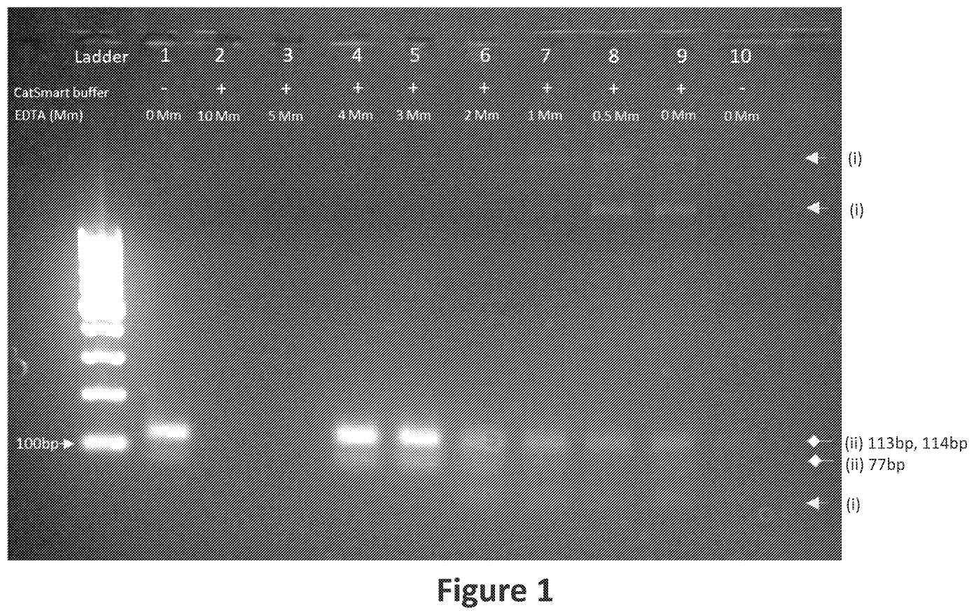

The reaction mix contained primers for three amplicons (Expected sizes—77 bp, 113 bp, 114 bp). The PCR products were analyzed on 1.8% agarose gel (FIG. 1, the lane numbering corresponds to the sample numbering above, 1-10). When undigested DNA (cfDNA in AVE buffer, without CutSmart buffer) was used, the PCR reaction was very efficient and produced the expected specific products (FIG. 1, lane 1). However, when mock digested DNA (with CutSmart buffer) was used, the efficiency of the PCR reaction was significantly reduced and resulted in amplification of nonspecific PCR products (FIG. 1, lane 9). When different concentrations of EDTA were added to the mock digested DNA, the reaction efficiency was gradually improved and non-specific product amplification were reduced. Efficient amplification and almost no amplification of nonspecific products (similar to the control undigested DNA in AVE buffer) achieved when 3-4 mM EDTA were added (FIG. 1 lanes 4-5). When more than 5 mM EDTA were added, the reaction efficiency dropped considerably with almost no amplification (FIG. 1 lanes 2-3).

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A method for profiling methylation of a DNA sample from a subject, the method comprising:
(i) subjecting the DNA sample to digestion with at least one methylation-sensitive restriction endonuclease in the presence of an amount of divalent cations sufficient to support cleavage of the DNA sample by the at least one methylation-sensitive restriction endonuclease, to obtain restriction endonuclease-treated DNA in which methylated sites are intact and unmethylated sites are cut;
(ii) adding a chelating agent to reduce the availability of the divalent cations by chelating the divalent cations; and
(iii) amplifying from the restriction endonuclease-treated DNA at least one restriction locus, wherein the amplification is carried out in a reaction mix comprising a chelating agent and divalent cations at a molar ratio of between 1:20 to 2:1, thereby generating an amplification product for said locus.

2. The method of claim 1, wherein the DNA sample digestion with at least one methylation-sensitive restriction endonuclease is carried out in a reaction mix comprising at least 8 mM divalent cations.

3. The method of claim 1, wherein the divalent cation is magnesium (Mg2+).

4. The method of claim 1, wherein the chelating agent is EDTA.

5. The method of claim 1, wherein the amplification step is carried out in a reaction mix comprising about 0.5-5 mM of the chelating agent.

6. The method of claim 1, wherein the digestion step is carried out in a reaction mix comprising less than 1 mM of the chelating agent.

7. The method of claim 1, wherein adding a chelating agent is performed by adding the restriction endonuclease-treated DNA of step (i) to reaction mix comprising the chelating agent.

8. The method of claim 1, wherein the sample is a plasma sample.

9. The method of claim 1, wherein the at least one methylation-sensitive restriction endonuclease is selected from the group consisting of AciI, HinP1I and HhaI.

10. The method of claim 1, wherein step (i) comprising digestion with the restriction enzymes HinP1I and HhaI or comprising digestion with the restriction enzymes HinP1I and AciI.

11. (canceled)

12. The method of claim 1, wherein the at least one restriction locus is a plurality of restriction loci.

13. The method of claim 1, wherein the at least one methylation-sensitive restriction endonuclease is a plurality of methylation-sensitive restriction endonucleases, and wherein the digestion with the plurality of methylation-sensitive restriction endonucleases is a simultaneous digestion.

14. The method of claim 1, wherein the at least one restriction locus is located within a CG-island.

15. The method of claim 1, wherein the amplification step comprises a step of co-amplification of at least one restriction locus and a control locus, thereby generating an amplification product for each locus.

16. The method of claim 1, wherein the method comprises a step of determining a signal intensity for each generated amplification product.

17. The method of any claim 1, wherein step (iii) is performed using real-time PCR.

18. The method of claim 1 any one of the preceding claims, wherein the DNA sample contains less than 5% ssDNA.

19. The method of claim 1, wherein the DNA sample is a cell-free DNA sample and optionally wherein an amount of cell-free DNA comprising 6,000 haploid equivalents is sufficient for the method.

20. The method of claim 19, wherein the cell-free DNA is extracted from a biological fluid sample.

21. (canceled)

22. The method of claim 20, wherein the cell-free DNA is plasma cell-free DNA, and wherein the amount of the cell-free DNA is an amount obtained from 8-10 ml of blood.

23. The method of claim 19, wherein the amount of cell-free DNA is between 10-400 ng.

24. The method of claim 1, wherein the DNA sample is from a subject suspected of having the disease and/or a subject at risk of developing the disease, and the method comprises detecting methylation changes and determining whether the DNA sample is a healthy or disease DNA sample, optionally wherein the disease is cancer.

25. (canceled)

26. A PCR reaction mix comprising between 0.5 mM and 20 mM chelating agent and a Taq polymerase.

27-29. (canceled)

30. A method for profiling methylation of a DNA sample from a subject, the method comprising:
(i) subjecting the DNA sample to digestion with at least one methylation-sensitive or methylation-dependent restriction endonuclease in the presence of an amount of divalent cations sufficient to support cleavage of the DNA sample by the at least one methylation-sensitive or methylation-dependent restriction endonuclease, to obtain restriction endonuclease-treated DNA;
(ii) adding a chelating agent to reduce the availability of the divalent cations by chelating the divalent cations; and
(iii) amplifying from the restriction endonuclease-treated DNA at least one restriction locus, wherein the amplification is carried out in a reaction mix comprising a chelating agent and a divalent cation at a molar ratio of between 1:20 to 2:1, thereby generating an amplification product for said locus.

31. A method for profiling methylation of a DNA sample from a subject, the method comprising:
(i) subjecting the DNA sample to digestion with at least one methylation-sensitive restriction endonuclease or at least one methylation-dependent restriction endonuclease in the presence of an amount of divalent cations sufficient to support cleavage of the DNA sample by the at least one methylation-sensitive or methylation-dependent restriction endonuclease, to obtain restriction endonuclease-treated DNA;
(ii) adding a chelating agent to reduce the availability of the divalent cations by chelating the divalent cations; and
(iii) preparing a sequencing library from the restriction endonuclease-treated DNA, wherein preparing the sequencing library comprises introducing adapter sequences into DNA fragments in the restriction endonuclease-treated DNA using PCR amplification, and wherein the PCR amplification is carried out in a buffer comprising a chelating agent and a divalent cation at a molar ratio of between 1:20 to 2:1.

32. A method for profiling methylation of a DNA sample from a subject, the method comprising:
(i) subjecting the DNA sample to digestion with at least one methylation-sensitive restriction endonuclease or at least one methylation-dependent restriction endonuclease in the presence of an amount of divalent cations sufficient to support cleavage of the DNA sample by the at least one methylation-sensitive or methylation-dependent restriction endonuclease, to obtain restriction endonuclease-treated DNA;
(ii) adding a chelating agent to reduce the availability of the divalent cations by chelating the divalent cations;
(iii) amplifying from the restriction endonuclease-treated DNA at least one restriction locus, wherein the amplification is carried out in a reaction mix comprising a chelating agent and a divalent cation at a molar ratio of between 1:20 to 2:1, thereby generating an amplification product for said locus; and
(iv) preparing a sequencing library from the amplification products, wherein preparing the sequencing library comprises adding sequencing adapters to the amplification products.

33. (canceled)

34. The method of claim 1, wherein the step of subjecting the DNA sample to digestion with at least one restriction endonuclease further comprises determining digestion efficacy, and proceeding to preparing a sequencing library if the digestion efficacy is above a predefined threshold.
